(19) United States
(12) Patent Application Publication
(10) Pub. No.: US 2021/0391036 A1
(43) Pub. Date: Dec. 16, 2021

(54) METHOD FOR PREDICTING A MOLECULAR WEIGHT DISTRIBUTION OF A BIOPOLYMER BLEND

(71) Applicant: ARC Medical Devices Inc., Richmond (CA)

(72) Inventors: Sailesh Haresh Daswani, Richmond (CA); Hoi Ting Wong, Richmond (CA)

(73) Assignee: ARC Medical Devices Inc., Richmond, BC (CA)

(21) Appl. No.: 17/260,281

(22) PCT Filed: Mar. 5, 2020
(86) PCT No.: PCT/CA2020/050294

§ 371 Date: Jan. 14, 2021

Related U.S. Application Data
(60) Provisional application No. 62/814,206, filed on Mar. 5, 2019.

(51) Int. Cl. G16C 20/30

(52) U.S. Cl. CPC G16C 20/30

(57) ABSTRACT

Methods, systems etc., for predicting and/or consistently obtaining uniform biopolymer compositions by blending a plurality of input biopolymer compositions with different molecular weight distributions, the blending based on concentration data as a function of molecular weight for the plurality of input biopolymer compositions.

### CLAIM FOR PRIORITY

The present application claims the benefit of co-pending U.S. provisional patent application No. 62/814,206 filed Mar. 5, 2019, the content of which is incorporated herein by reference in their entirety.

### BACKGROUND

Polymers are high molecular weight compounds that may be naturally occurring (biopolymers) or are synthesized by a polymerization reaction. A repeating structural unit of a polymer is called a monomer unit. Polymers can be delineated by their degree of polymerization, molecular weight distribution, tacticity, copolymer distribution, degree of branching, end-groups, crosslinks, crystallinity and/or thermal properties. Polymers display a wide array of characteristics in solution with respect to their solubility, viscosity and/or gelation.

The molecular weight distribution of polymers can be delineated using various metrics such as the peak molecular weight (PMW), weight average molecular weight (WAMW), number average molecular weight (NAMW), full width half maximum (FWHM) and polydispersity index (PDI). The molecular weight distribution of a polymer is indicative of certain properties of the polymer, for example, the solubility and/or viscosity of the polymer in solution.

Some processes of producing synthetic polymers result in polymers with a unimodal molecular weight distribution of relatively low polydispersity. Naturally occurring polymers, on the other hand, can be found to have erratic and unpredictable molecular weight distributions with undesirable polydispersity. Such biopolymers include for example, polysaccharides such as starch, glycogen, cellulose, chitin, arabinoxylan, xyloglucan, alginate, laminarin, fucan, xanthan gum, dextran, welan gum, gellan gum, guar gum, diotan gum and pullulan.

Many medical and/or surgical uses have been found for biopolymers, some of which are related to a specific molecular weight fraction or segment of the biopolymer. The utility of medically relevant fractions or segments of biopolymers has led to numerous methods of producing, purifying and/or extracting the medically relevant fractions or segments, for example methods using membrane dialysis, tangential flow filtration and controlled degradation to obtain a desired molecular weight segment of the biopolymer. These methods suffer from natural variation in input material, resulting in low yields of the desired biopolymer molecular weight segment. These challenges in the consistent preparation of desired biopolymer molecular weight segments become commensurately greater as the molecular weight and polydispersity of the biopolymer increase. Thus, there has gone unmet a need for providing biopolymers derived from natural sources but having desired molecular weight segments and/or unimodal distributions. The present systems and methods, etc., provide these and/or other advantages.

### SUMMARY

Methods, systems etc., are provided for predicting and/or consistently obtaining uniform biopolymer compositions by blending a plurality of input biopolymer compositions with different molecular weight distributions, the blending based on concentration data as a function of molecular weight for the plurality of input biopolymer compositions.

The present methods, systems, etc., provide methods for predicting and/or consistently obtaining uniform biopolymer compositions having desired molecular weight distributions, as well as compositions comprising such uniform biopolymer compositions with desired molecular weight distributions and methods of use of such compositions. The obtained uniform biopolymer compositions can themselves be used as an input biopolymer composition in other processes, for example, purification processes, chemical modification processes, molecular weight fractionation processes, etc.

The present systems, devices and methods, etc., provide methods for predicting a molecular weight distribution of a blended biopolymer composition can comprise:
providing a plurality of input biopolymer compositions having substantially differing molecular weight distributions;
obtaining concentration data as a function of molecular weight for each of the plurality of input biopolymer compositions using chromatography;
for each input biopolymer composition, normalizing the concentration data as a function of molecular weight to provide normalized concentration data; and
combining the normalized concentration data for each input biopolymer composition at a selected number of matching molecular weight values to obtain a predicted biopolymer molecular weight distribution.

In some aspects, the present systems, devices and methods, etc., provide for predicting a unimodal molecular weight distribution of a blended biopolymer composition. Such methods can comprise, for example:
providing a first plurality of input biopolymer compositions having substantially differing molecular weight distributions;
obtaining concentration data as a function of molecular weight for each of the first plurality of input biopolymer compositions using chromatography;
for each of the first plurality of input biopolymer compositions, normalizing the concentration data as a function of molecular weight to provide normalized concentration data;
determining molecular weight distribution standard deviations and number average molecular weights for each of the first plurality of input biopolymer compositions; identifying from among the first plurality of input biopolymer compositions a base input biopolymer composition;
selecting from among the first plurality of biopolymer compositions a second plurality of input biopolymer compositions having number average molecular weights that differ from the number average molecular weight of the base input biopolymer composition by less than twice the molecular weight distribution standard deviation of the base input biopolymer composition; and
combining the normalized concentration signal data of each of the second plurality of input biopolymer compositions and the base input biopolymer composition at a selected number of matching molecular weight values to obtain a predicted unimodal biopolymer molecular weight distribution.

In some aspects, the present systems, devices and methods, etc., provide for obtaining a unimodal blended biopolymer composition from first and second input biopolymer compositions. Such methods can comprise, for example:
determining for the first and second input biopolymer compositions respective first and second number average molecular weight and standard deviation;
selecting from among the first and second standard deviation the smaller standard deviation;
blending together the first and second input biopolymer compositions only if the difference between the first and second number average molecular weights can be less than about twice the smaller standard deviation, and
if the first and second input biopolymer compositions can be blended together, obtaining the unimodal blended biopolymer composition.

In some aspects, the present systems, devices and methods, etc., provide for obtaining a unimodal blended biopolymer composition from at least two input biopolymer compositions. Such methods can comprise, for example:
determining for each input biopolymer composition respective number average molecular weight and molecular weight distribution standard deviation data;
selecting from among the input biopolymer compositions a base input biopolymer composition; and
blending with the base input biopolymer composition only the input biopolymer compositions that have a number average molecular weight that differs from the base input biopolymer composition number average molecular weight by at most about two times the molecular weight distribution standard deviation of the base input biopolymer composition.

The concentration data can comprise a measurement signal indicating the concentration of input biopolymer composition as a function of molecular weight. The measurement signal can comprise, for example, a refractive index measurement signal, an ultra-violet absorption measurement signal, an infrared absorption measurement signal, a fluorescence measurement signal, an electrochemical measurement signal, a conductivity measurement signal, a chemiluminescence measurement signal, a radiometric measurement signal, or an evaporative light scattering measurement signal.

The chromatography can be, for example, gel permeation chromatography, size exclusion chromatography, gel electrophoresis chromatography, or ion exchange chromatography. The chromatography can comprise first collecting concentration data as a function of retention time and converting the retention time values to molecular weight values using a molecular weight-retention time calibration curve.

Combining the normalized concentration data can comprise, for example, first subjecting the concentration data to a baseline correction at a predetermined threshold before combining the normalized concentration data, combining the normalized concentration data at a predetermined weighting. The predetermined weighting can be based, for example, on multiple simulations can comprise combining the normalized concentration data for the plurality of input biopolymer compositions to provide at least one of a desired weigh average molecular weight, number average molecular weight and peak molecular weight; by solving for at least one of a desired weight average molecular weight, number average molecular weight and peak average molecular weight; or, be based on a predetermined formula.

In certain embodiments, the methods further can comprise blending the plurality of input biopolymer compositions according to the predetermined weighting to obtain a blended biopolymer composition and/or, before obtaining the concentration data, removing unwanted impurities from the input biopolymer composition.

These and other aspects, features and embodiments are set forth within this application, including the following Detailed Description and attached drawings. Unless expressly stated otherwise, all embodiments, aspects, features, etc., can be mixed and matched, combined and permuted in any desired manner.

### BRIEF DESCRIPTION OF THE DRAWINGS

FIG. 1 provides a flow chart depicting an exemplary method for predicting the molecular weight distribution of a blended biopolymer composition obtained by blending a plurality of input biopolymer compositions.

FIG. 2 provides a flow chart depicting an exemplary method for predicting the unimodal molecular weight distribution of a blended biopolymer composition obtained by blending a plurality of input biopolymer compositions.

FIG. 3 provides a flow chart depicting an exemplary method for forming a unimodal blended biopolymer composition from two input, biopolymer compositions.

FIG. 4 shows a calibration graph and curve fit of molecular weight to gel permeation chromatography retention time using dextran as calibrant.

FIG. 5 shows refractive index signal versus molecular weight graphs of two feedstock fucoidan compositions, a predicted unimodal blended fucoidan composition comprising 60% of the first feedstock fucoidan and 40% of the second feedstock fucoidan and a real unimodal blended composition comprising 60% of the first feedstock fucoidan with 40% of the second feedstock fucoidan.

The drawings, including the flow charts, present exemplary embodiments of the present disclosure. The drawings are not necessarily to scale and certain features may be exaggerated or otherwise represented in a manner to help illustrate and explain the present systems, methods, etc. Actual embodiments of the systems, methods, etc., herein may include further or different features or steps not shown in the drawings. The exemplifications set out herein illustrate embodiments of the systems, methods, etc., in one or more forms, and such exemplifications are not to be construed as limiting the scope of the disclosure in any manner. The embodiments herein are not exhaustive and do not limit the disclosure to the precise form disclosed, for example in the following detailed description.

### DETAILED DESCRIPTION

Methods, systems etc., are provided for predicting and consistently obtaining a uniform biopolymer composition having a desired molecular weight distribution by blending a plurality of input biopolymer compositions, the blending method based on biopolymer concentration data as a function of molecular weight for the plurality of input biopolymer compositions.

Suitable biopolymers for use with the methods herein include without limitation starch, glycogen, cellulose, chitin, arabinoxylan, xyloglucan, alginate, laminarin, fucan, xanthan gum, dextran, welan gum, gellan gum, guar gum, diotan gum and pullulan. The methods are discussed herein using fucoidan compositions as examples of more general fucan and other biopolymer compositions, and are discussed herein using gel permeation chromatography with refractive index detection as an exemplary method for obtaining biopolymer concentration data as a function of molecular weight.

Briefly, fucans (including fucoidan) are sulfated polysaccharides, are typically derived from natural sources and have high polydispersity. In general terms, this means that fucans are molecules made up of a number of monomer or monosaccharide groups, and also have sulfur atoms attached to the sugar groups. The main monosaccharide group is called “fucose”, which is sugar that has 6 carbon atoms and has the chemical formula C₆H₁₂O₅. “Fucoidan” (or fucoidin) indicates fucans derived from brown algae (seaweed). Fucans can contain a mixture of other monomer or monosaccharide units, for example a mixture of monosaccharides such as xylose, galactose, glucose, glucuronic acid and/or mannose. Although fucans are currently derived from natural sources such as the brown algae (seaweeds), sea cucumbers, etc., “fucan” includes polymer molecules having the chemical and structural motifs of the fucans as discussed herein regardless of the ultimate source(s) of the fucans. Further, the methods, etc., herein apply to any relevant polydisperse input compositions, whether or not naturally derived or fucan-based.

In some embodiments, for example where naturally sourced biopolymer compositions, including feedstock fucan compositions, are used as input biopolymer compositions for the analyses and blending herein, the input biopolymer compositions may be dissolved in water and pre-filtered through a suitable pre-filter to remove undesired particulate matter. The input polydisperse biopolymer compositions may also be pre-treated to remove components other than the desired biopolymer.

Exemplary methods for the determination of the concentration of an input biopolymer composition as a function of molecular weight, or the molecular weight distribution, are size exclusion chromatography, gel permeation chromatography, gel electrophoresis and ion exchange chromatography. In gel permeation chromatography, the concentration of biopolymer in an eluting solvent is monitored continuously with a detector. Suitable detector types include without limitation ultra-violet/visible (UV/Vis) absorption detectors, refractive index (RI) detectors, infrared (IR) absorption detectors, fluorescence (FLR) detectors, electrochemical detectors, conductivity detectors, chemiluminescence detectors, radioactivity or radiometric detectors and evaporative light scattering (ELS) detectors.

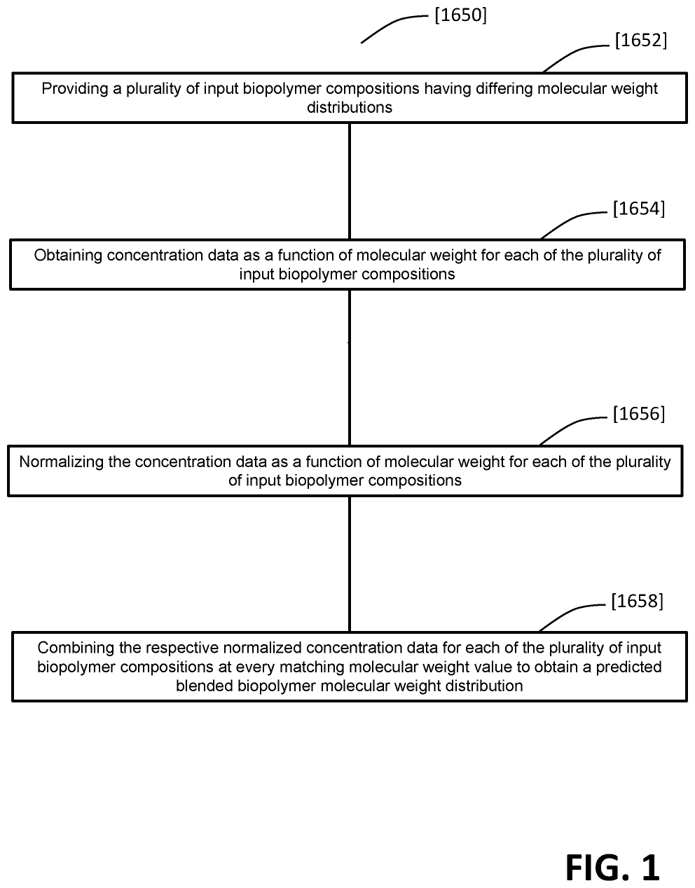

The flow chart of FIG. 1 depicts an exemplary method [1650] for predicting the molecular weight distribution of a blended biopolymer composition obtained by blending a plurality of input biopolymer compositions, the method comprising: providing [1652] a plurality of input biopolymer compositions having differing molecular weight distributions; obtaining [1654] concentration data as a function of molecular weight for each of the plurality of input biopolymer compositions; normalizing [1656] the concentration data as a function of molecular weight for each of the plurality of input biopolymer compositions; and combining [1658] the respective normalized concentration data for each of the plurality of input biopolymer compositions at every matching molecular weight value to obtain a predicted blended biopolymer molecular weight distribution.

The concentration data may be, for example, any one or more of a refractive index measurement signal, ultra-violet absorption measurement signal, infrared absorption measurement signal, fluorescence measurement signal, electrochemical measurement signal, conductivity measurement signal, chemiluminescence measurement signal, radiometric measurement signal and evaporative light scattering measurement signal.

The concentration data as a function of molecular weight may be obtained [1654], for example, by any one of gel permeation chromatography, size exclusion chromatography, gel electrophoresis or ion exchange chromatography.

Obtaining [1654] the concentration data as a function of molecular weight may comprise first collecting the concentration data as a function of retention time and converting the retention time to molecular weight through the use of a molecular weight versus retention time calibration curve.

Method [1650] may, if desired, further comprise pre-treating the input biopolymer compositions before obtaining [1654] concentration data as a function of molecular weight for each of the plurality of input biopolymer compositions. The pre-treating may comprise diafiltrating the input biopolymer composition with distilled water to desalt the input biopolymer composition. The pre-treating may comprise diafiltrating the input biopolymer across a tangential flow filtration (TFF) filter having a molecular weight cutoff (MWCO) based on select impurities to be removed. Method [1650] may further comprise pre-filtering the input biopolymer compositions before obtaining [1654] concentration data as a function of molecular weight for each of the plurality of input biopolymer compositions to remove undesired particular matter.

The combining [1658] of the respective normalized concentration data of each of the plurality of input biopolymer compositions at every selected matching molecular weight value may comprise subjecting the concentration data to a baseline correction at a predetermined threshold prior to combining the respective normalized concentration data.

Combining [1658] the respective normalized concentration data of each of the plurality of input biopolymer compositions at every matching molecular weight value may involve combining the concentration data on the basis of a predetermined weighting, the predetermined weighting configured to result in a desired predicted biopolymer molecular weight distribution. In some embodiments, the predetermined weighting may be based on a pre-calibration of the above method for different input biopolymer compositions and predicted biopolymer compositions. In other embodiments, the predetermined weighting may be based on a predetermined formula. In yet other embodiments, the predetermined weighting may be based on multiple simulations of combining the normalized concentration data of each of the plurality of biopolymer compositions until a desired weight average molecular weight (WAMW), number average molecular weight (NAMW) or peak molecular weight (PMW) in the predicted molecular weight distribution is obtained. In yet other embodiments, the predetermined weighting may be obtained by solving for a weighting that would result in a desired weight average molecular weight (WAMW), number average molecular weight (NAMW) or peak molecular weight (PMW) in the resulting predicted molecular weight distribution.

Many input biopolymer compositions, for example, feedstock fucoidan compositions, have large polydispersity, for example a polydispersity greater than 4.0, 5.0 or 6.0. One condition relating to blending input biopolymer compositions may be that the blending would provide a resulting molecular weight distribution with a single peak, also known as a “unimodal distribution”. For example, given two different input molecular weight distributions, the blending of the two input molecular weight distributions will result in a unimodal distribution if the difference in the number average molecular weights of the two input molecular weight distributions is at most about twice the molecular weight value of the smaller molecular weight distribution standard deviation of the two input molecular weight distributions.

In some embodiments, the input biopolymer composition with the smallest molecular weight distribution standard deviation may be identified from among a first plurality of input fucoidan compositions. The term “base input biopolymer composition” indicates the input biopolymer composition with the smallest molecular weight distribution standard deviation. A second plurality of input biopolymer compositions, being a subset of the first plurality of input biopolymer compositions, may be identified from among the first plurality of input biopolymer compositions to have NAMW values that differ from the NAMW of the base input biopolymer composition by less than twice the molecular weight value of the molecular weight distribution standard deviation of the base input biopolymer composition.

With the predicted molecular weight distribution of the blended biopolymer composition obtained, the weight average molecular weight, peak molecular weight, number average molecular weight, full width half maximum and polydispersity of the blend may be determined by using formulae for each respective attribute. The creation of a template capable of taking x-variable, for example molecular weight, versus concentration for multiple input biopolymer compositions and manipulating these datasets to calculate the weight average molecular weight, peak molecular weight, number average molecular weight, full width half maximum and polydispersity of the blended biopolymer compositions resulting from a given blend ratio of a number of input biopolymer compositions eliminates the need for rigorous bench-top trial and error testing during the blending of input biopolymer compositions.

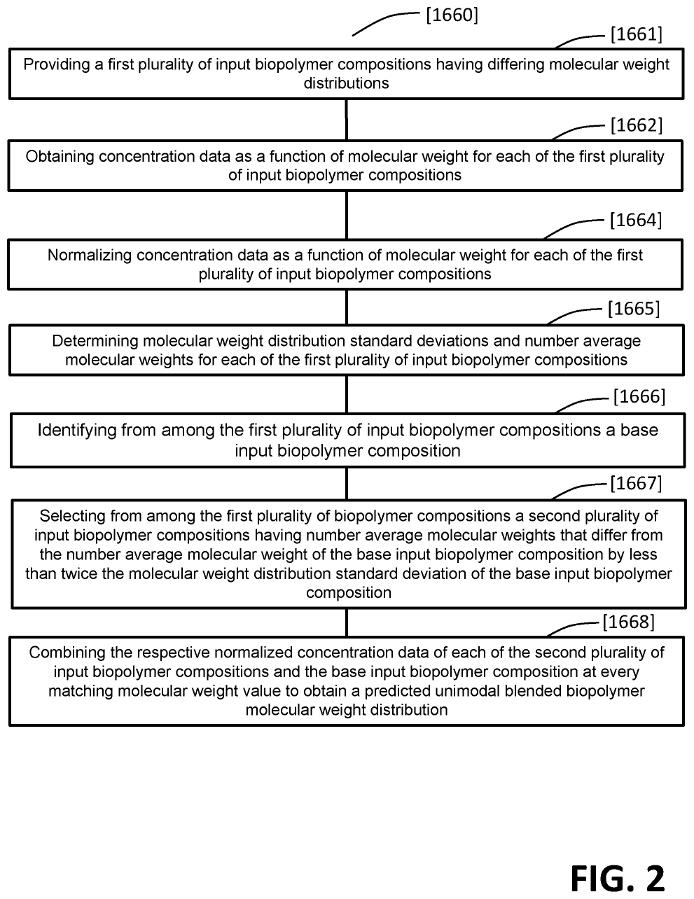

The flow chart of FIG. 2 depicts a further exemplary method [1660] for predicting the unimodal molecular weight distribution of a blended biopolymer composition obtained by blending a plurality of input biopolymer compositions, the method comprising: providing [1661] a first plurality of input biopolymer compositions having differing molecular weight distributions; obtaining [1662] concentration data as a function of molecular weight for each of the first plurality of input biopolymer compositions; normalizing [1664] concentration data as a function of molecular weight for each of the first plurality of input biopolymer compositions; determining [1665] molecular weight distribution standard deviations and number average molecular weights for each of the first plurality of input biopolymer compositions; identifying [1666] from among the first plurality of input biopolymer compositions a base input biopolymer composition; selecting [1667] from among the first plurality of biopolymer compositions a second plurality of input biopolymer compositions having number average molecular weights that differ from the number average molecular weight of the base input biopolymer composition by less than twice the molecular weight distribution standard deviation of the base input biopolymer composition; and combining [1668] the respective normalized concentration data of each of the second plurality of input biopolymer compositions and the base input biopolymer composition at every selected matching molecular weight value to obtain a predicted unimodal blended biopolymer molecular weight distribution.

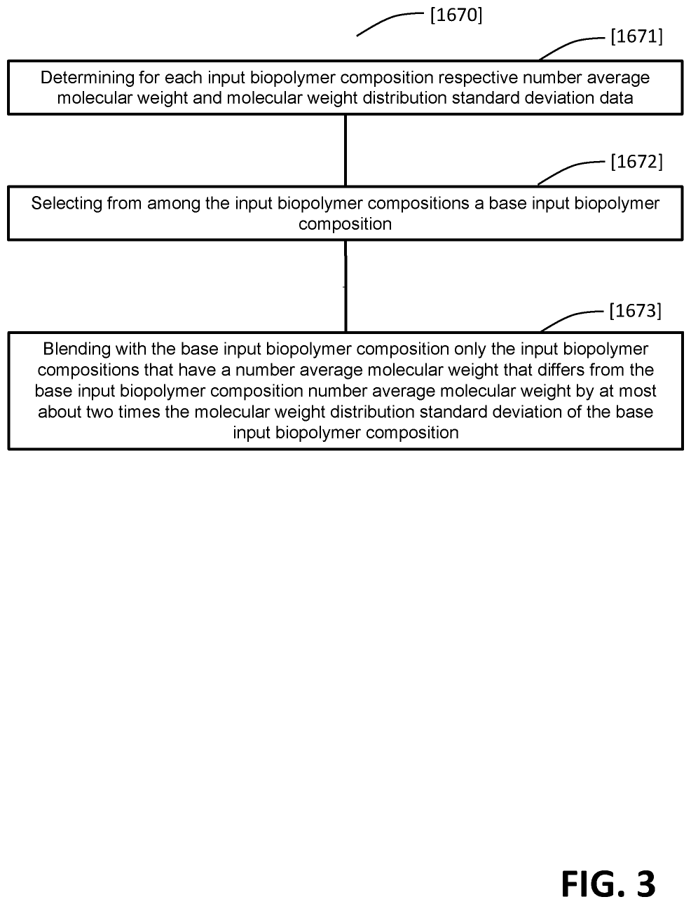

The flow chart of FIG. 3 depicts another exemplary method [1670] for obtaining a unimodal blended biopolymer composition by combining at least two input biopolymer compositions comprising: determining [1671] for each input biopolymer composition respective number average molecular weight and molecular weight distribution standard deviation data; selecting [1672] from among the input biopolymer compositions a base input biopolymer composition; and blending [1673] with the base input biopolymer composition only the input biopolymer compositions that have a number average molecular weight that differs from the base input biopolymer composition number average molecular weight by at most about two times the molecular weight distribution standard deviation of the base input biopolymer composition.

### EXAMPLES

### Example 1: Blending of Two Input Biopolymer Compositions after Prediction of the Blending Outcome Based on a Predetermined Weighting

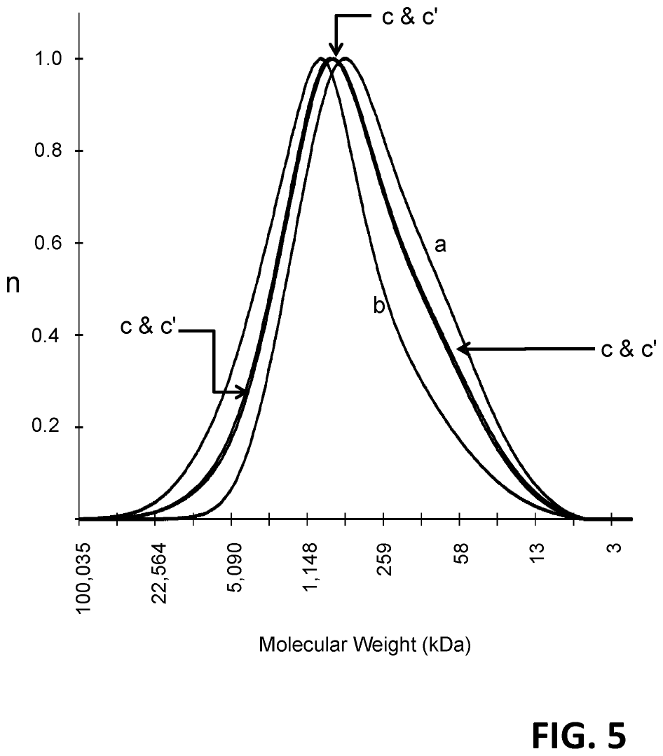

Fucoidan was selected as the biopolymer of interest and the above method shown in FIG. 2 was applied to two different feedstock fucoidan compositions. The first feedstock fucoidan composition molecular weight distribution is shown in FIG. 5 as curve “a”, and the second feedstock fucoidan composition molecular weight distribution is shown in FIG. 5 as curve “b”. Both feedstock fucoidan compositions were analyzed by gel permeation chromatography with a refractive index detector to obtain their concentration data as a function of molecular weight.

The number average molecular weight and molecular weight distribution standard deviation were calculated for the two feedstock fucoidan compositions. Curve “b”, or the second feedstock fucoidan composition, was found to have a smaller molecular weight distribution standard deviation. The number average molecular weight of the two feedstock fucoidan compositions differed by less than two times the molecular weight distribution standard deviation of the second feedstock fucoidan composition. A target weight average molecular weight for the blended biopolymer composition was selected and a predicted weighting of 60% of the first feedstock fucoidan composition and 40% of the second feedstock fucoidan composition was predicted to result in the desired target weight average molecular weight. The normalized refractive index signals of curve “a” and curve “b” were added together in accordance with the predicted weighting to obtain a predicted unimodal blended fucoidan composition curve and the molecular weight distribution attributes were calculated and shown in table 1 below.

60 g of the first feedstock fucoidan composition was blended with 40 g of the second feedstock fucoidan composition in 1 L of deionized water to result in a real unimodal blended fucoidan composition. An aliquot of the real unimodal blended fucoidan composition was analyzed via gel permeation chromatography with a refractive index detector to obtain concentration data as a function of molecular weight.

All gel permeation chromatography analyses for molecular weight determination were conducted using the following column configuration: Ultrahydrogel® 2000/Ultrahydrogel® Linear in series with an Ultrahydrogel® guard. The mobile phase was 0.1M sodium nitrate run at 0.6 mL/min unless stated otherwise. The column and detector were held at 30° C. unless stated otherwise. Detection was by means of a Waters 2414 refractive index detector. The concentration data is in this case a refractive index signal denoted by the symbol “n” in the figures.

Samples run were quantified against a standard curve comprising of traceable standards from the American Polymer Standards Corporation: Dextran 3755 kDa (peak molecular weight=2164 kDa), Dextran 820 kDa (peak molecular weight=745 kDa), Dextran 760 kDa (peak molecular weight=621 kDa), Dextran 530 kDa (peak molecular weight=490 kDa), Dextran 225 kDa (peak molecular weight=213 kDa), Dextran 150 kDa (peak molecular weight=124 kDa), Dextran 55 kDa (peak molecular weight=50 kDa) and Dextran 5 kDa (peak molecular weight=4 kDa), the peak molecular weights of these standards being between about 4 kDa and about 2,200 kDa. The standard curve used may, for example, include Dextran 3755 kDa and between 4 to 7 additional traceable standards discussed herein.

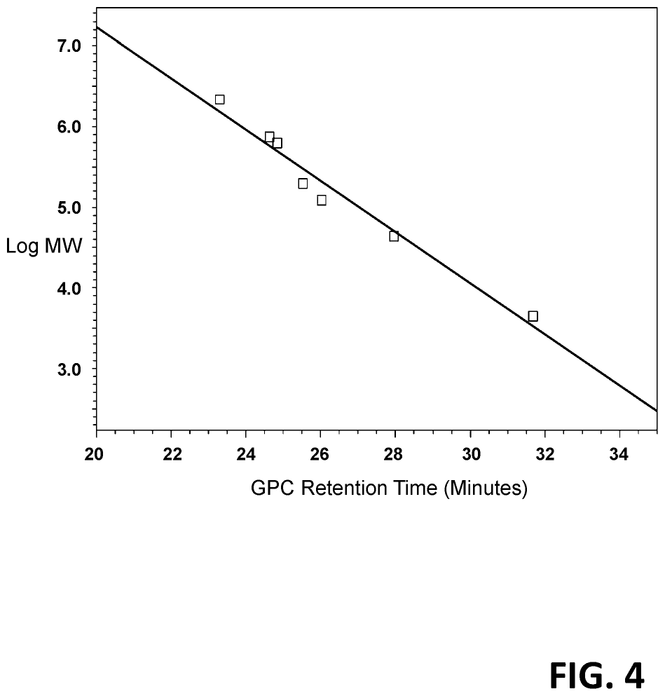

A linear curve fit was made to the data based on a log MW versus GPC retention time plot. This curve is provided in FIG. 4. The curve provides molecular weight as a function of GPC retention time and allows the conversion of GPC retention times to corresponding molecular weights, as discussed in the methods disclosed herein.

A molecular weight stated for a fucan/fucoidan biopolymer herein is a value of molecular weight about which there will always be a distribution of molecules of higher and lower molecular weights, increasing or decreasing in amount or percentage as the molecular weight increases or decreases away from the specified molecular weight. The distribution may, but is not required to, have a generally Gaussian or distorted Gaussian shape.

The curve resulting from the gel permeation chromatography of the real unimodal blended composition was compared to the predicted unimodal blended fucoidan composition curve. Molecular weight distribution attributes for the two curves are presented in Table 1 in which some of the curves in FIG. 5 are identified. FIG. 5 shows normalized curves resulting from the gel permeation chromatography of the first (a) and second (b) feedstock fucoidan compositions, a normalized curve (c) of the predicted unimodal blended composition comprising of 60% of the first feedstock fucoidan composition and 40% of the second feedstock fucoidan composition, and a normalized curve (c′) resulting from the gel permeation chromatography of the real unimodal blended fucoidan composition comprising of 60% of the first feedstock fucoidan composition and 40% of the second feedstock fucoidan composition, the vertical axis in each being the refractive index, n.

Abbreviations in the table below:

TABLE 1
Predicted and real blended fucoidan composition
molecular weight distribution attributes

| | Real unimodal | Predicted unimodal | Percent |
|---|---|---|---|
| | blend (c′) | blend (c) | difference |
| WAMW (Da) | 1152502 | 1125000 | 2% |
| PMW (Da) | 730966 | 688691 | 6% |
| NAMW (Da) | 169832 | 162951 | 4% |
| PDI | 6.79 | 6.90 | 2% |

Peak molecular weight = PMW
Weight average molecular weight = WAMW
Number average molecular weight = NAMW
Polydispersity index = PDI

Table 1 and FIG. 5 demonstrate the prediction capability of the methods herein. For example, the curves of predicted versus real unimodal blended fucoidan compositions are virtually indistinguishable. In other words, the calculated molecular weight distribution attributes in the predicted unimodal blend fucoidan composition agree with the calculated molecular weight distribution attributes of the real unimodal blend fucoidan composition, particularly for WAMW, NAMW and PDI where the difference is less than 5%. This is within the established accuracy of gel permeation chromatography.

The prediction of molecular weight distribution attributes such as weight average molecular weight, number average molecular weight, polydispersity and peak molecular weight of a predicted blended biopolymer composition removes the need for tedious trial and error experiments to consistently produce uniform biopolymer compositions.

The present application is further directed to compositions made according to the methods, systems etc., discussed herein as well as to methods of using the compositions created herein, and to systems and devices configured to perform the methods herein and consistently obtain uniform biopolymer compositions with desired molecular weight distributions.

### REFERENCE NUMERAL LIST

1650 A method for predicting the molecular weight distribution of a blended biopolymer composition obtained by blending a plurality of input biopolymer compositions
1652 Providing a plurality of input biopolymer compositions having differing molecular weight distributions
1654 Obtaining concentration data as a function of molecular weight for each of the plurality of input biopolymer compositions
1656 Normalizing the concentration data as a function of molecular weight for each of the plurality of input biopolymer compositions
1658 Combining the respective normalized concentration data for each of the plurality of input biopolymer compositions at every matching molecular weight value to obtain a predicted blended biopolymer molecular weight distribution
1660 A method for predicting the unimodal molecular weight distribution of a blended biopolymer composition obtained by blending a plurality of input biopolymer compositions.
1661 Providing a first plurality of input biopolymer compositions having differing molecular weight distributions
1662 Obtaining concentration data as a function of molecular weight for each of the first plurality of input biopolymer compositions
1664 Normalizing concentration data as a function of molecular weight for each of the first plurality of input biopolymer compositions
1665 Determining molecular weight distribution standard deviations and number average molecular weights for each of the first plurality of input biopolymer compositions
1666 Identifying from among the first plurality of input biopolymer compositions a base input biopolymer composition
1667 Selecting from among the first plurality of biopolymer compositions a second plurality of input biopolymer compositions having number average molecular weights that differ from the number average molecular weight of the base input biopolymer composition by less than twice the molecular weight distribution standard deviation of the base input biopolymer composition
1668 Combining the respective normalized concentration data of each of the second plurality of input biopolymer compositions and the base input biopolymer composition at every matching molecular weight value to obtain a predicted unimodal blended biopolymer molecular weight distribution
1670 A method for obtaining a unimodal blended biopolymer composition by combining at least two input biopolymer compositions
1671 Determining for each input biopolymer composition respective number average molecular weight and molecular weight distribution standard deviation data
1672 Selecting from among the input biopolymer compositions a base input biopolymer composition
1673 Blending with the base input biopolymer composition only the input biopolymer compositions that have a number average molecular weight that differs from the base input biopolymer composition number average molecular weight by at most about two times the molecular weight distribution standard deviation of the base input biopolymer composition

All terms used herein are used in accordance with their ordinary meanings unless the context or definition clearly indicates otherwise. Also unless expressly indicated otherwise, in the specification the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

Unless otherwise stated, adjectives herein such as “substantially” and “about” that modify a condition or relationship characteristic of a feature or features of an embodiment, indicate that the condition or characteristic is defined to within tolerances that are acceptable for operation of the embodiment for an application for which it is intended.

The scope of the present methods, compositions, systems, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “step” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion and figures herein.

1. A method for predicting a molecular weight distribution of a blended biopolymer composition comprising:
providing a plurality of input biopolymer compositions having substantially differing molecular weight distributions;
obtaining concentration data as a function of molecular weight for each of the plurality of input biopolymer compositions;
for each input biopolymer composition, normalizing the concentration data as a function of molecular weight to provide normalized concentration data; and
combining the normalized concentration data for each input biopolymer composition at a selected number of matching molecular weight values to obtain a predicted biopolymer molecular weight distribution corresponding to the plurality of input biopolymer compositions.

2. The method of claim 1 wherein the concentration data comprises a refractive index measurement signal indicating the concentration of input biopolymer composition as a function of molecular weight.

3. The method of claim 1 wherein the concentration data comprises at least one of a) an ultra-violet absorption measurement signal indicating the concentration of input biopolymer composition as a function of molecular weight, b) an infrared absorption measurement signal indicating the concentration of input biopolymer composition as a function of molecular weight, c) a fluorescence measurement signal indicating the concentration of input biopolymer composition as a function of molecular weight, d) an electrochemical measurement signal indicating the concentration of input biopolymer composition as a function of molecular weight, e) a conductivity measurement signal indicating the concentration of input biopolymer composition as a function of molecular weight, f) chemiluminescence measurement signal indicating the concentration of input biopolymer composition as a function of molecular weight, g) a radiometric measurement signal indicating the concentration of input biopolymer composition as a function of molecular weight, or h) an evaporative light scattering measurement signal indicating the concentration of input biopolymer composition as a function of molecular weight.

4-10. (canceled)

11. The method of claim 1 wherein obtaining the concentration data comprises size exclusion chromatography.

12. The method of claim 1 wherein obtaining the concentration data comprises gel electrophoresis chromatography.

13. The method of claim 1 wherein obtaining the concentration data comprises ion exchange chromatography.

14. The method of any of claims 11 to 13 wherein the chromatography comprises first collecting concentration data as a function of retention time and converting the retention time values to molecular weight values using a molecular weight-retention time calibration curve.

15. The method of claim 1 wherein combining the normalized concentration data comprises combining the normalized concentration data at a predetermined weighting.

16. The method of claim 14 wherein the predetermined weighting is based on multiple simulations comprising combining the normalized concentration data for the plurality of input biopolymer compositions to provide at least one of a desired weight average molecular weight, number average molecular weight and peak molecular weight.

17. The method of claim 14 wherein the predetermined weighting is obtained by solving for at least one of a desired weight average molecular weight, number average molecular weight and peak average molecular weight.

18. The method of claim 14 wherein the predetermined weighting is based on a predetermined formula.

19. The method of claim 14 further comprising blending the plurality of input biopolymer compositions according to the predetermined weighting to obtain a blended biopolymer composition.

20. The method of claim 1 wherein the method further comprises, before obtaining the concentration data, removing unwanted impurities from the input biopolymer composition.

21. The method of claim 1 wherein the predicted biopolymer molecular weight distribution is within 6% of a real blend of the input biopolymer compositions having the substantially differing molecular weight distributions.

22. The method of claim 1 wherein the predicted biopolymer molecular weight distribution is within 4% of a real blend of the input biopolymer compositions having the substantially differing molecular weight distributions.

23. A method for predicting a unimodal molecular weight distribution of a blended biopolymer composition comprising:
providing a first plurality of input biopolymer compositions having substantially differing molecular weight distributions;
obtaining concentration data as a function of molecular weight for each of the first plurality of input biopolymer compositions;
for each of the first plurality of input biopolymer compositions, normalizing the concentration data as a function of molecular weight to provide respective normalized concentration data;
determining molecular weight distribution standard deviations and number average molecular weights for each of the first plurality of input biopolymer compositions;
identifying from among the first plurality of input biopolymer compositions a base input biopolymer composition;
selecting from among the first plurality of biopolymer compositions a second plurality of input biopolymer compositions having number average molecular weights that differ from the number average molecular weight of the base input biopolymer composition by less than twice the molecular weight distribution standard deviation of the base input biopolymer composition; and
combining the respective normalized concentration data of each of the second plurality of input biopolymer compositions and the base input biopolymer composition at a selected number of matching molecular weight values to obtain a predicted unimodal blended biopolymer molecular weight distribution.

24. The method of claim 23 wherein the predicted unimodal biopolymer molecular weight distribution is within 6% of a real blend of the base input biopolymer composition and the second plurality of input biopolymer compositions.

25. The method of claim 23 wherein the predicted unimodal biopolymer molecular weight distribution is within 4% of a real blend of the base input biopolymer composition and the second plurality of input biopolymer compositions.

26. A method for obtaining a unimodal blended biopolymer composition from at least two input biopolymer compositions, the method comprising:
determining for each input biopolymer composition respective number average molecular weight and molecular weight distribution standard deviation data;
selecting from among the input biopolymer compositions a base input biopolymer composition; and
blending with the base input biopolymer composition only the input biopolymer compositions that have a number average molecular weight that differs from the base input biopolymer composition number average molecular weight by at most about two times the molecular weight distribution standard deviation of the base input biopolymer composition.
